(19) United States
(12) Patent Application Publication
(10) Pub. No.: US 2014/0128601 A1
(43) Pub. Date: May 8, 2014

(54) PROCESS FOR OBTAINING RIVAROXABAN AND INTERMEDIATE THEREOF

(71) Applicants: Xavier Berzosa Rodríguez, L'hospitalet De Llobregat (ES); Francisco Marquillas Olondriz, Barcelona (ES); Amadeo Llebaria Soldevilla, Sant Cugat Del Valles (ES); Carme Serra Comas, L'Hospitalet De Llobregat (ES)

(72) Inventors: Xavier Berzosa Rodríguez, L'hospitalet De Llobregat (ES); Francisco Marquillas Olondriz, Barcelona (ES); Amadeo Llebaria Soldevilla, Sant Cugat Del Valles (ES); Carme Serra Comas, L'Hospitalet De Llobregat (ES)

(73) Assignee: INTERQUIM, S.A., Sant Cugat Del Vallès (ES)

(21) Appl. No.: 14/118,853

(22) PCT Filed: May 18, 2012
(86) PCT No.: PCT/EP2012/059272

§ 371 Date: Jan. 6, 2014

(30) Foreign Application Priority Data
May 20, 2011 (ES) P 201130817

(51) Int. Cl. C07D 413/14; C07D 413/10

(52) U.S. Cl. CPC C07D 413/14; C07D 413/10
USPC 544/137

(57) ABSTRACT

This invention relates to a procedure for obtaining a thiophene-2-carboxamide compound, specifically rivaroxaban, which comprises the (i) fragmentation of the N═C bond of a compound of formula 23 where R1 is selected among hydrogen, halogen, and (C1-C6)alkyl; and (ii) acylation of the resulting intermediate with 5-chloro-tiofen-2-carbonyl chloride in a solvent medium, in the presence of a base. The invention also relates to the compounds of formula 23 and their use in the obtention of rivaroxaban.

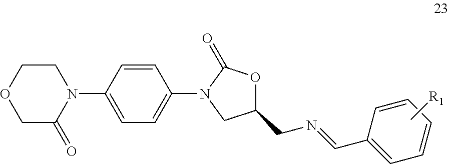

This invention relates to a procedure for obtaining a thiophene-2-carboxamide, specifically rivaroxaban (RVX). Rivaroxaban (RVX) corresponds chemically to (S)-5-chloro-N-((2-oxo-3-(4-(3-oxomorpholin)phenyl)oxazolidine-5-yl)methyl)thiophene-2-carboxamide, and is an inhibitor of the active form of coagulation factor X (factor Xa). It is used clinically as an anticoagulant. Its structural formula is:

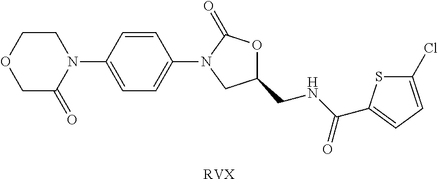

### STATE OF THE PRIOR ART

Several synthetic routes have been described to date for obtaining rivaroxaban (RVX). All of them share the use of aniline 1 as key intermediate.

The first route described is that of product U.S. Pat. No. 7,157,456B2, which was subsequently optimised in U.S. Pat. No. 7,351,823B2, for an escalation in the order of 2 Kg (FIG. 1).

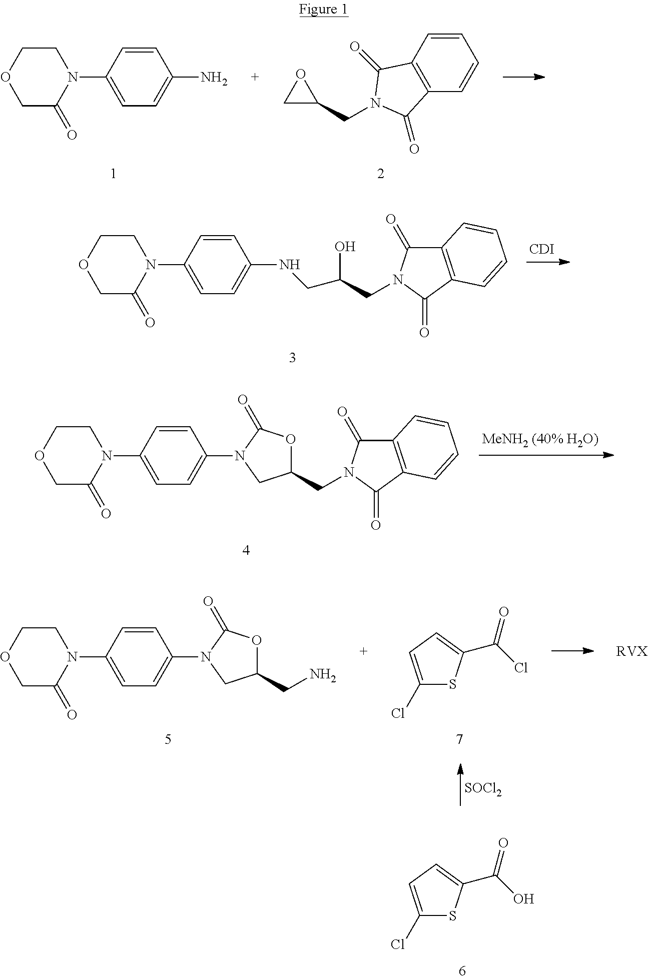

The global yield of the optimised process is 62%, including a final purification step by recrystallisation in acetic acid. This procedure is also described in several articles: Journal of Medicinal Chemistry 2005, 48, 5900-5908, Drugs of the Future 2006, 31, 484-493; and IP.com Journal 2009, 9(4A), 10.

The second route described is claimed in patent application WO2004060887A1. The procedure described is exemplified for a scale of 25 g (FIG. 2).

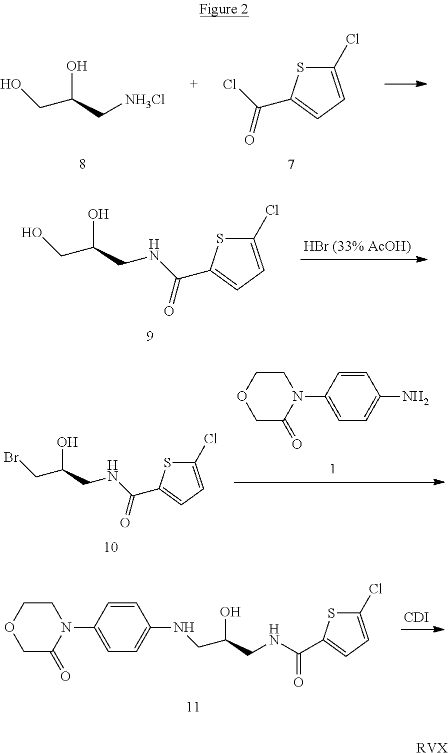

The global yield of the process is 37%.

Finally, U.S. Pat. No. 7,816,355B1 exemplifies the procedure for obtaining rivaroxaban (at a scale of 1 mg) through intermediate 15 with a global yield of 26% (FIG. 3).

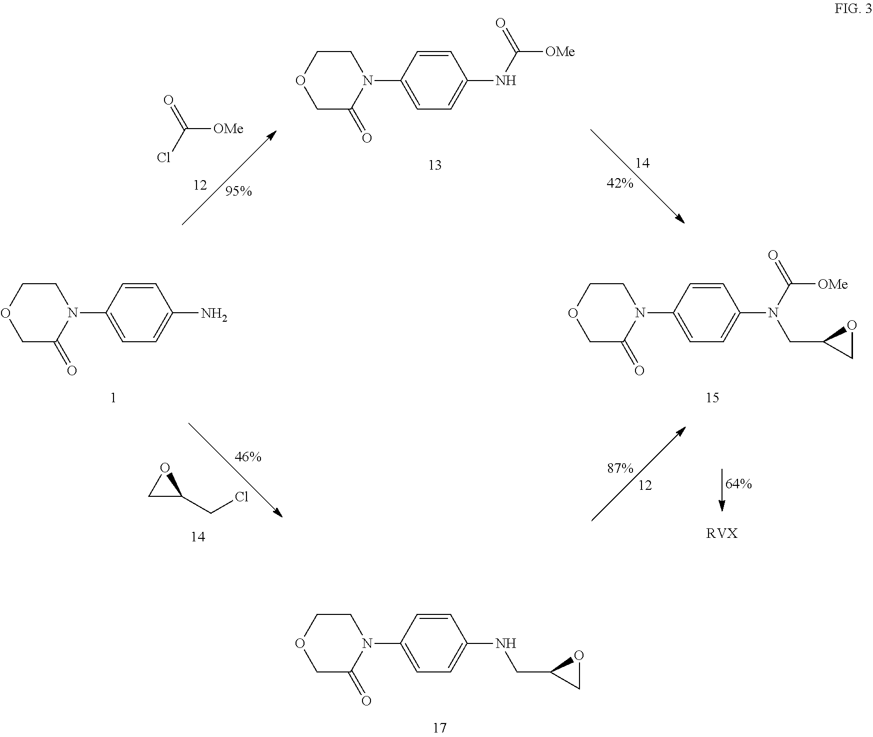

In summary, the routes known to date show generally low yields, and in some case expensive starting materials are used, such as (S)—N-glycidyl phthalimide (2). The low yields and the use of expensive starting materials involve high production costs.

We therefore conclude, according to the procedures described to date, that there is a need for having a new procedure for obtaining rivaroxaban industrially with an adequate yield, while avoiding the use of expensive starting materials.

### SUMMARY OF THE INVENTION

The invention provides a new advantageous industrial procedure for obtaining rivaroxaban (RVX), according to the chart of FIG. 4.

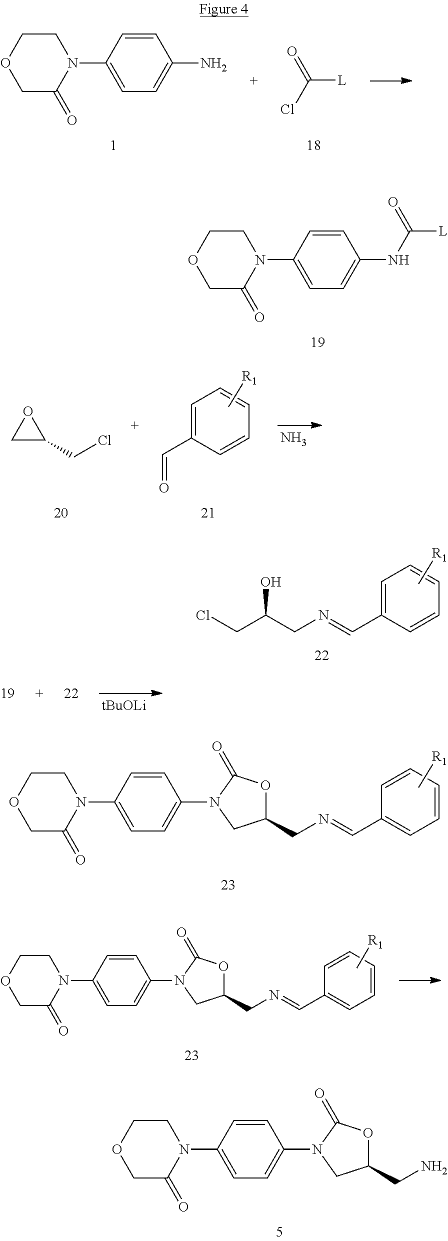

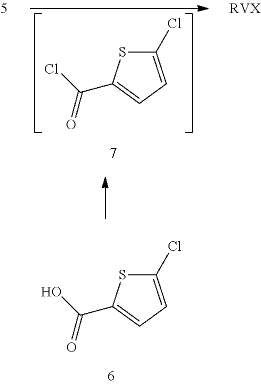

The global yield of the process is 70%-85%. This yield improvement associated with the use of cheap reagents replacing (S)—N-glycidyl phthalimide (2) leads to achieving a significant cost reduction vs the state of the art through the procedure of this invention.

### DETAILED DESCRIPTION OF THE INVENTION

The object of this invention is to provide a procedure for obtaining a thiophene-2-carboxamide, specifically rivaroxaban (RVX).

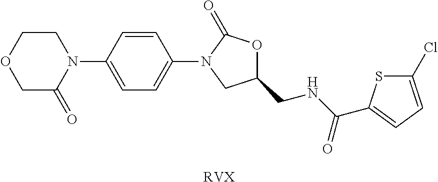

and synthesis intermediates, comprising the following steps:
(i) fragmentation of the N═C bond of a compound of formula 23

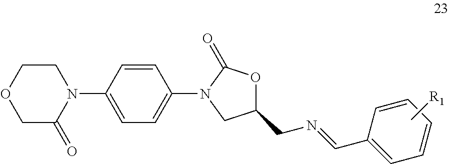

where R₁ is selected among hydrogen, halogen, and (C₁-C₆)alkyl; and
(ii) acylation of the resulting intermediate of formula 5

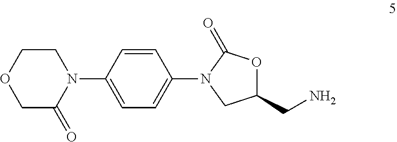 [SMILES: NC[C@H]1CN(c2ccc(N3CCOCC3=O)cc2)C(=O)O1]

with the acid chloride of formula 7

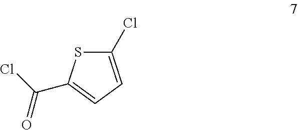

in a solvent medium, in the presence of a base.

In a preferred embodiment R₁ is hydrogen or 4-chloro.

Step (i) can be performed by hydrolysis with an acid in a solvent medium, followed by neutralisation with a base; or by reaction with a primary amine in an optional solvent medium.

In a particular embodiment, step (i) comprises the hydrolysis of a compound of formula 23 with an acid in a solvent medium, followed by neutralisation with a base.

In a preferred embodiment, by way of illustration rather than limitation, in step (i) the acid is hydrochloric acid, hydrobromic acid, acetic acid, or sulphuric acid, the solvent medium is an ester, an ether, an aromatic hydrocarbon, a ketone, a halogenated hydrocarbon or water or mixtures thereof, and the base is triethylamine, diisopropylethylamine, sodium hydroxide, potassium hydroxide, sodium carbonate, potassium carbonate, sodium bicarbonate, potassium bicarbonate, calcium hydroxide, calcium carbonate, or calcium bicarbonate or mixtures thereof.

More preferably, the solvent medium used in step (i) is ethyl acetate, methyl acetate, isopropyl acetate, tetrahydrofuran, dioxane, diethyl ether, methyl t-butyl ether, diisopropyl ether, dibutyl ether, toluene, o-xylene, m-xylene, p-xylene, acetone, methyl ethyl ketone, methyl isobutyl ketone, dichloromethane, dichloroethane or water or mixtures thereof.

In another particular embodiment, step (i) comprises the reaction of a compound of formula 23 with a primary amine, in an optional solvent medium.

In a preferred embodiment, by way of illustration rather than limitation, in step (i) the primary amine is amongst others methylamine, ethylamine, propylamine or isobutylamine; and the optional solvent medium is an ester, an ether, an aromatic hydrocarbon, an aliphatic hydrocarbon, a ketone, an alcohol, a halogenated hydrocarbon or water or mixtures thereof.

More preferably, the optional solvent medium used in step (i) is ethyl acetate, methyl acetate, isopropyl acetate, tetrahydrofuran, dioxane, diethyl ether, methyl t-butyl ether, diisopropyl ether, dibutyl ether, toluene, o-xylene, m-xylene, p-xylene, heptane, hexane, acetone, methyl ethyl ketone, methyl isobutyl ketone, methanol, ethanol, n-butanol, tert-butanol, dichloromethane, dichloroethane or water or mixtures thereof.

In another preferred embodiment, in step (ii) the solvent medium is an ester, an ether, an aromatic hydrocarbon, a ketone, a halogenated hydrocarbon or water or mixtures thereof, and the base is triethylamine, diisopropylethylamine, sodium hydroxide, potassium hydroxide, sodium carbonate, potassium carbonate, sodium bicarbonate, potassium bicarbonate, calcium hydroxide, calcium carbonate or calcium bicarbonate or mixtures thereof.

In another embodiment, more preferred in step (ii), the solvent medium is ethyl acetate, methyl acetate, isopropyl acetate, tetrahydrofuran, dioxane, diethyl ether, methyl t-butyl ether, diisopropyl ether, dibutyl ether, toluene, o-xylene, m-xylene, p-xylene, acetone, methyl ethyl ketone, methyl isobutyl ketone, dichloromethane, dichloroethane or water or mixtures thereof.

It is also an object of the invention herein to provide a procedure for obtaining a compound of formula 23

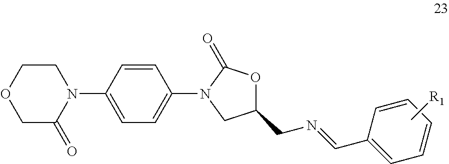

where R₁ is selected among hydrogen, halogen, and (C₁-C₆)alkyl, comprising the reaction of a compound of formula 19

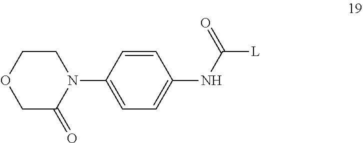

where L is a leaving group, with a compound of formula 22

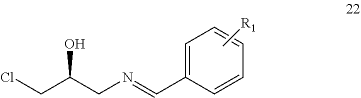

where R₁ has the same meaning as in 23, in the presence of a base and in a solvent medium.

In a preferred embodiment, the leaving group L is O—R₂, where R₂ is (C₁-C₁₅)alkyl, aryl, aryl-(C₁-C₄)alkyl, heteroaryl or heteroaryl-(C₁-C₄)alkyl, so that aryl means a phenyl or naphthyl group, that can be optionally substituted. Similarly, heteroaryl means a monocyclic aromatic ring of 5 or 6 members or bicyclic of 8 to 10 members, containing 1 to 4 heteroatoms selected independently among nitrogen, oxygen and sulphur, and that can be optionally substituted. An aromatic ring will be defined in this invention as an unsaturated ring with all atoms with sp² hybridation and where the total number of pi electrons is 4n+2, where n is an integer of value 1 or 2. In a heteroaryl-(C₁-C₄)alkyl group, the heteroaryl group may be bound to the rest of the molecule through any heteroatom available in the aromatic ring. Examples of heteroaryl groups include, amongst others, furan, imidazole, pyrazole, pyrrole, thiophene, pyridine, pyrimidine, benzimidazole, benzofuran, benzothiophene, indole, quinoline, and quinoxaline. In this invention “optionally substituted” is defined as the optional presence of 1 to 3 substituents selected independently among (C₁-C₄)alkyl, halogen, (C₁-C₄)alkoxy, —CF₃, —CN, —NO₂, —OH, —COR′, —OCOR′, —CO₂R′, —CONR′R″, —NR′R″, —NR″CO₂R′, —SOR′ and —SO₂R′, where R′ and R″ independently represent hydrogen, (C₁-C₄)alkyl, phenyl, or naphthyl.

In another preferred embodiment, the base is lithium tert-butoxide or lithium tert-amoxide and the solvent medium, by way of illustration rather than limitation, comprises an ether, an aliphatic hydrocarbon, a halogenated hydrocarbon or a nitrile, or mixtures thereof.

More preferably, the solvent medium comprises tetrahydrofuran, dioxane, diethyl ether, methyl t-butyl ether, diisopropyl ether, dibutyl ether, heptane, hexane, dichloromethane, dichloroethane, acetonitrile, propionitrile, butyronitrile, or benzonitrile or mixtures thereof.

Furthermore, the object of this invention is to provide a procedure for obtaining thiophene-2-carboxamide of formula RVX

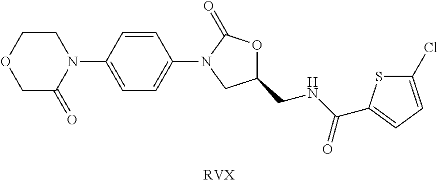

comprising the following steps:
(i) reaction of a compound of formula 19

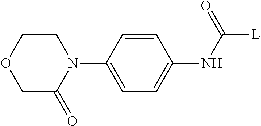

where L is a leaving group, with a compound of formula 22

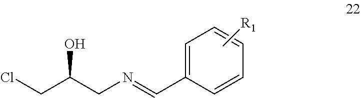

where R₁ is selected among hydrogen, halogen and (C₁-C₆)alkyl, in the presence of a base and in a solvent medium;
(i) fragmentation of the N═C bond of a compound formed in step (i), of formula 23

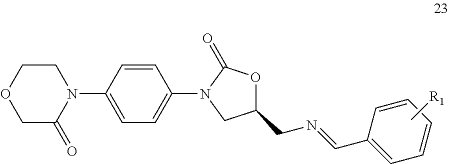

where R₁ has the same meaning as in 22; and
(ii) acylation of the intermediate resulting of formula 5

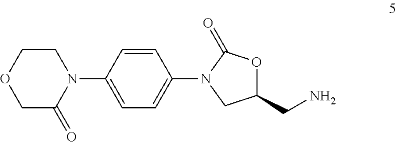 [SMILES: NC[C@H]1CN(c2ccc(N3CCOCC3=O)cc2)C(=O)O1]

with the acid chloride of formula 7

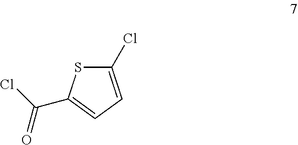

in a solvent medium, in the presence of a base; so that preferred embodiments of the procedures are the aforementioned definitions of the leaving group L, of the base and the solvent medium in step (i); of the solvent medium and the base in step (iii); as well as of the different methods for obtaining amine 5 from the compound of formula 23 according to step (ii), i.e. hydrolysis with an acid in a solvent medium, followed by neutralisation with a base and reaction with a primary amine, in an optional solvent medium; and each of the preferred embodiments of the aforementioned methods.

Also the object of this invention is to provide a compound of formula 23

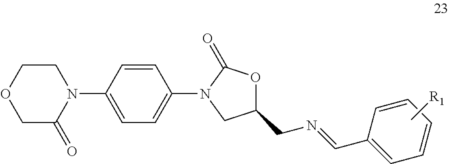

where R₁ is selected among hydrogen, halogen and (C₁-C₆)alkyl, with hydrogen and 4-chloro being preferred.

Finally, the object of this invention is also the use of a compound of formula 23, preferably when R₁ is hydrogen or 4-chloro, for the synthesis of rivaroxaban (RVX).

In this invention the term (C₁₋ₓ)alkyl refers to a linear or branched alkyl chain containing 1 to x carbon atoms. For instance, a (C₁₋₁₅)alkyl group relates to a linear or branched alkyl chain containing 1 to 15 carbon atoms. Therefore, a (C₁₋₁₅)alkyl group includes, amongst others, methyl, ethyl, propyl, isopropyl, butyl, isobutyl, sec-butyl, tert-butyl, n-pentyl, n-hexyl, n-heptyl, n-octyl, n-nonyl, n-decyl, n-undecyl, n-dodecyl, n-tridecyl, n-tetradecyl, and n-pentadecyl. When R₂ is (C₁-C₁₅)alkyl, n-undecyl, n-dodecyl, n-tridecyl, n-tetradecyl, and n-pentadecyl groups are particularly preferred.

In this invention the term (C₁₋₄)alkoxy refers to a linear or branched alkoxy chain containing 1 to 4 carbon atoms. Therefore, a (C₁₋₄)alkoxy group includes methoxy, ethoxy, propoxy, isopropoxy, butoxy, isobutoxy, sec-butoxy, and tert-butoxy.

It must be noted that in this invention there is no need for isolating the intermediate 5, resulting of oxazolidinone 23, for its reaction with acid chloride 7 leading to RVX.

By way of illustration, Table 1 summarises the yields obtained for the reactions performed as named in the chart of FIG. 4. In this invention, two chloroformates 18 and two different benzaldehydes 21 were used (FIG. 5), to obtain the compounds 23a (R₁═Cl) or 23b (R₁═H), respectively.

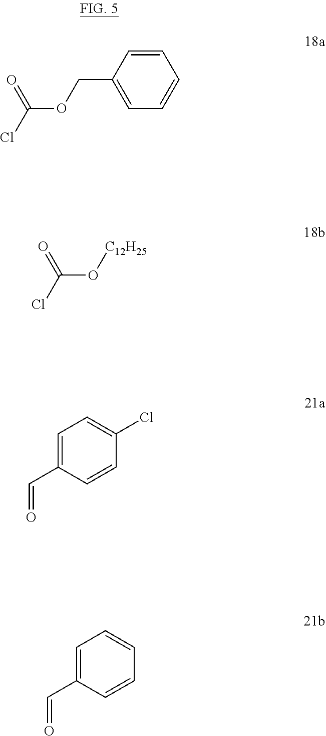

TABLE 1

| Reagent | Yield | Product | Example |
|---|---|---|---|
| 18a + 1 | 96% | 19a | 1 |
| 18b + 1 | 94% | 19b | 2 |
| 21a + 20 | 59% | 22a | 3 |
| 21b + 20 | 75% | 22b | 4 |
| 19a + 22a | 68% | 23a | 5 |
| 19b + 22a | 90% | 23a | 5 |
| 19a + 22b | 88% | 23b | 6 |
| 19b + 22b | 83% | 23b | 6 |
| 23a | 51% | 5 | 7a |
| 23a | 98.5% | 5 | 7b |
| 23a | 99.7% | 5 | 7d |
| 23a + 7 | 83% | RVX | 8b1 |
| 23b + 7 | 72% | RVX | 8b2 |
| 5 + 7 | 96% | RVX | 8b3.a |
| 5 + 7 | 100% | RVX | 8b3.b |
| 5 + 7 | 90% | RVX | 8b3.c |

In view of these results we can see that RVX is obtained through the process outlined herein, with a global yield (starting from aniline 1) of 70%-85%.

The invention is illustrated below with the following examples, which must not be understood in any case as limiting the scope of this invention.

### EXAMPLES

### Example 1

### Obtaining benzyl (4-(3-oxomorpholin)phenyl)carbamate (19a)

Prepare a dissolution of 20 g of 4-(4-aminophenyl)morpholin-3-one (1) (104 mmol) in a mixture of 439 mL of acetone and 220 mL of water. Cool the solution to 0° C. and add 18.5 g of sodium carbonate (175 mmol). After 10 min slowly add over the mixture 15.9 mL of benzyl chloroformate (18a) (111 mmol) and stir at room temperature for 4 h. Filter the solid obtained and wash with water. Vacuum dry at room temperature, obtaining 32.5 g (96% yield) of the intended product (19a) as a white solid. Mp: 198° C. MS: m/z=349 (M+Na). IR (cm⁻¹)=1650, 1725. ¹H NMR (500 MHz, CDCl₃) δ 7.38 (m, 7H), 7.23 (d, J=8.7 Hz, 2H), 6.87 (s, 1H), 5.20 (s, 2H), 4.32 (s, 2H), 4.04-3.97 (m, 2H), 3.75-3.68 (m, 2H).

### Example 2

### Obtaining dodecyl (4-(3-oxomorpholin)phenyl)carbamate (19a)

Prepare a dissolution of 1 g of 4-(4-aminophenyl)morpholin-3-one (1) (5.2 mmol) in a mixture of 22 mL of acetone and 11 mL of water. Cool the solution to 0° C. and add 0.93 g of sodium carbonate (8.8 mmol). After 10 min slowly add over the mixture 1.5 mL of dodecyl chloroformate (18b) (5.6 mmol) and stir at room temperature for 4 h. Filter the solid obtained and wash with water. Redissolve the solid in dichloromethane, add anhydrous sodium carbonate, filter, and remove the dissolvent at reduced pressure. Vacuum dry at room temperature, obtaining 1.98 g (94% yield) of the intended product (19b) as a white solid. Mp: 152° C. MS: m/z=405 (M+1). IR (cm⁻¹)=1650, 1725. ¹H NMR (500 MHz, CDCl₃) δ 7.44 (d, J=8.3 Hz, 2H), 7.26 (s, 2H), 6.71 (s, 1H), 4.35 (s, 2H), 4.17 (t, J=6.7 Hz, 2H), 4.04 (dd, J=5.7, 4.3 Hz, 2H), 3.78-3.72 (m, 2H), 1.72-1.64 (m, 2H), 1.42-1.23 (m, 18H), 0.89 (t, J=6.9 Hz, 3H)

### Example 3

### Obtaining (S)-1-Chloro-3-[(4-chlorobenzylidine)-amino]-propan-2-ol (22a)

Prepare a solution of 1 g of 4-chlorobenzaldehyde (21a) (7 mmol) in 4.3 mL of methyl t-butyl ether (MTBE). Add 0.7 mL of 30% aqueous ammonia (11 mmol) and stir the mixture for 15 min at room temperature. Slowly add 0.56 mL of (S)-(+)-epichlorhydrin (20) (7 mmol) and stir at room temperature for 40 min. Heat to 40° C. and stir at this temperature for another 18 h.

Separate the phases and remove the solvent from the organic phase at reduced pressure.

Reconstitute with 2 mL of MTBE and 2 mL of hexane and cool to 0-5° C. for 2 h. Sow the solution with crystals of the intended product and, after 1 h at 0-5° C., filter the crystals obtained and wash them with cold hexane. Vacuum dry the solid at room temperature, obtaining 0.84 g of the intended product. Sow again the stock water and, after 18 h at 0-5° C., filter the crystals obtained, wash them with cold hexane and vacuum dry at room temperature, obtaining 0.12 g of the intended product. Join the solids obtained, resulting in 0.96 g (59% yield) of the intended product (22a) as a white solid. Mp=89-95° C. IR (cm⁻¹): 1670. ¹H NMR (500 MHz, CD₃OD) δ 8.33 (s, 1H), 7.75 (d, J=8.3 Hz, 2H), 7.44 (d, J=8.3 Hz, 2H), 4.07 (p, J=5.5 Hz, 1H), 3.84 (dd, J=12.2, 4.8 Hz, 1H), 3.72-3.63 (m, 2H), 3.60 (dd, J=11.2, 5.6 Hz, 1H). MS: m/z=232 (M). [α]_{D}=a/c I=−12.58±0.10° g⁻¹ mL dm⁻¹ (c=1.51 g/100 mL in chloroform).

### Example 4

### Obtaining (S)-3-(benzylideneamino)-1-chloro-propan-2-ol (22b)

Prepare a 2.6 mL solution of benzaldehyde (21b) (26 mmol) in 15.4 mL of MTBE. Add 2.6 mL of 30% aqueous ammonia (38 mmol) and stir the mixture for 15 min at room temperature. Slowly add 2 mL of (S)-(+)-epichlorhydrin (20) (26 mmol) and stir at room temperature for 40 min. Heat to 40° C. and stir at this temperature for another 18 h. Separate the phases and reduce the solvent of the organic phase to half at reduced pressure. Add 10 mL of hexane and cool at 0° C. for 45 min. Filter the precipitate obtained and wash with cold hexane (3×5 mL. Vacuum dry the solid at room temperature, obtaining 3.8 g (75% yield) of the intended product (22b) as a white solid. Mp=46° C. IR (cm⁻¹): 1698.8. ¹H NMR (500 MHz, CDCl₃) δ 8.36 (s, 1H), 7.74 (dd, J=7.7, 1.4 Hz, 2H), 7.43 (d, J=7.3 Hz, 3H), 4.14 (p, J=5.6 Hz, 1H), 3.83 (ddd, J=12.6, 5.0, 1.1 Hz, 1H), 3.77 (ddd, J=12.7, 5.9, 1.1 Hz, 1H), 3.67 (dd, J=5.4, 4.8 Hz, 2H). [α]_{D}=α/c I=+1.72±0.06° g⁻¹ mL dm⁻¹ (c=0.86 mg/100 mL in chloroform).

### Example 5

### Obtaining (S)-4-[4-(5-{[(4-chlorobenzylidene)-amino]-methyl}-2-oxo-oxazolidine-3-yl)-phenyl]-morpholin-3-one (23a)

From 19a and 22a:

To a mixture containing 0.32 g of benzyl (4-(3-oxomorpholin)phenyl)carbamate (19a) (1 mmol) and 0.2 g of t-BuOLi (2.5 mmol) add 10 mL of dichlorometane and then 0.25 g of (S)-1-chloro-3-[(4-chlorobenzylidene)-amino]-propan-2-ol (22a) (1 mmol). Heat the mixture at reflux for 24 h. Extract with water (1×10 mL; 1×5 mL). Evaporate dichloromethane and reconstitute with 5 mL of dichloromethane and 15 mL of iPrOH. Concentrate to a volume of 10 mL and cool at −10/−20° C. Filter the solid obtained, wash with cold iPrOH and vacuum dry at room temperature, obtaining 0.27 g (67% yield) of the intended product (23a) as yellow solid. Mp=157-160° C. IR (cm⁻¹): 2850.62, 1725, 1651. ¹H NMR (500 MHz, CDCl₃) δ 8.35 (s, 1H), 7.64 (d, J=8.4 Hz, 2H), 7.59 (d, J=8.9 Hz, 2H), 7.36 (dd, J=12.6, 8.7 Hz, 4H), 4.98 (dq, J=10.2, 5.1 Hz, 1H), 4.34 (s, 2H), 4.14 (ddd, J=14.6, 13.0, 7.4 Hz, 2H), 4.06-4.01 (m, 2H), 3.95 (ddd, J=36.6, 13.1, 4.6 Hz, 2H), 3.78-3.73 (m, 2H). MS: m/z=420 (M+Li), 833 (2M+Li), 1248 (3M+Li). [α]_{D}=α/c I=−215.57±0.39° g⁻¹ mL dm⁻¹ (c=0.86 mg/100 mL in chloroform).

From 19a and 22a:

To a mixture containing 0.32 g of dodecyl (4-(3-oxomorpholin)phenyl)carbamate (19b) (0.78 mmol) and 0.16 g of t-BuOLi (2 mmol) add 6 mL of dichloromethane and then 0.2 g de (S)-1-chloro-3-[(4-chlorobenzylidine)-amino]-propan-2-ol (22a) (0.86 mmol). Heat the mixture at reflux for 1.5 days. Extract with water (1×10 mL; 1×5 mL). Evaporate dichloromethane and reconstitute with 5 mL of dichloromethane and 15 mL of iPrOH. Concentrate to a volume of 10 mL and cool at −10/−20° C. Filter the solid obtained, wash with cold iPrOH and vacuum dry at room temperature, obtaining 0.29 g (90% yield) of the intended product (23a) as a yellow solid. Mp=157-160° C. IR (cm⁻¹): 2850.62, 1725, 1651. ¹H NMR (500 MHz, CDCl₃) δ 8.35 (s, 1H), 7.64 (d, J=8.4 Hz, 2H), 7.59 (d, J=8.9 Hz, 2H), 7.36 (dd, J=12.6, 8.7 Hz, 4H), 4.98 (dq, J=10.2, 5.1 Hz, 1H), 4.34 (s, 2H), 4.14 (ddd, J=14.6, 13.0, 7.4 Hz, 2H), 4.06-4.01 (m, 2H), 3.95 (ddd, J=36.6, 13.1, 4.6 Hz, 2H), 3.78-3.73 (m, 2H). MS: m/z=420 (M+Li), 833 (2M+Li), 1248 (3M+Li). [α]_{D}=α/c I=−215.57±0.39° g⁻¹ mL dm⁻¹ (c=0.86 mg/100 mL in chloroform).

### Example 6

### Obtaining (S)-4-(4-{5-[(benzylidene-amino)-methyl]-2-oxo-oxazolidine-3-yl}-phenyl)-morpholin-3-one (23b)

From 19a and 22b:

To a mixture containing 0.26 g of benzyl (4-(3-oxomorpholin)phenyl)carbamate (19a) (0.78 mmol) and 0.16 g of t-BuOLi (2.0 mmol) add 6 mL of dichloromethane and then 0.17 g de (S)-3-(benzylidene-amino)-1-chloro-propan-2-ol (22b) (0.86 mmol). Heat the mixture at reflux for 24 h. Extract with water (1×10 mL; 1×5 mL). Evaporate dichloromethane and reconstitute with 5 mL of dichloromethane and 15 mL of iPrOH. Concentrate to a volume of 10 mL and cool at −10/−20° C. Filter the solid obtained, wash with cold iPrOH and vacuum dry at room temperature, obtaining 0.26 g (88% yield) of the intended product (23b) as a yellow solid. Mp=136° C. IR (cm⁻¹): 2874.46, 2851.06; 1727.59, 1649.82. ¹H NMR (500 MHz, CDCl₃) δ 8.40 (s, 1H), 7.73-7.68 (m, 2H), 7.60 (d, J=8.9 Hz, 2H), 7.47-7.37 (m, 3H), 7.34 (d, J=8.9 Hz, 2H), 4.98 (td, J=10.9, 5.5 Hz, 1H), 4.34 (s, 2H), 4.20-4.09 (m, 2H), 4.05-4.02 (m, 2H), 3.96 (ddd, J=18.4, 11.2, 6.4 Hz, 2H), 3.78-3.72 (m, 2H).

From 19a and 22b:

To a mixture containing 0.32 g of dodecyl (4-(3-oxomorpholin)phenyl)carbamate (19b) (0.78 mmol) and 0.16 g of t-BuOLi (2.0 mmol) add 6 mL of dichloromethane and then 0.17 g of (S)-3-(benzylidene-amino)-1-chloro-propan-2-ol (22b) (0.86 mmol). Heat the mixture at reflux for 1.5 days. Extract with water (1×10 mL; 1×5 mL). Evaporate dichloromethane and reconstitute with 5 mL of dichloromethane and 15 mL of iPrOH. Concentrate to a volume of 10 mL and cool at −10/−20° C. Filter the solid obtained, wash with cold iPrOH and vacuum dry at room temperature, obtaining 0.25 g (83% yield) of the intended product (23b) as a yellow solid. Mp=136° C. IR (cm⁻¹): 2874.46, 2851.06; 1727.59, 1649.82. ¹H NMR (500 MHz, CDCl₃) δ 8.40 (s, 1H), 7.73-7.68 (m, 2H), 7.60 (d, J=8.9 Hz, 2H), 7.47-7.37 (m, 3H), 7.34 (d, J=8.9 Hz, 2H), 4.98 (td, J=10.9, 5.5 Hz, 1H), 4.34 (s, 2H), 4.20-4.09 (m, 2H), 4.05-4.02 (m, 2H), 3.96 (ddd, J=18.4, 11.2, 6.4 Hz, 2H), 3.78-3.72 (m, 2H).

### Example 7

### Obtaining (S)-4-[4-(5-aminomethyl-2-oxo-oxazolidine-3-yl)phenyl]morpholin-3-one (5)

7a)

Mix 0.5 g of (S)-4-[4-(5-{[(4-chlorobenzylidine)-amino]-methyl}-2-oxo-oxazolidine-3-yl)phenyl]-morpholin-3-one (23a) (1.2 mmol), 17.5 mL of water, and 17.5 mL of AcOEt. Then add 0.2 mL of 37% HCl in water (2.4 mmol) and stir at room temperature for 2 hours. Separate the phases. Extract the aqueous phase with 35 mL of AcOEt. Discard the organic phases. Basify the aqueous phase to pH=10 and extract with dichloromethane (5×5 mL). Join the organic phases, dry with anhydrous Na₂SO₄, filter, and remove the solvent at reduced pressure. 178 mg (51% yield) of the intended product (5) are obtained as a white solid. Mp=145-147° C. IR (cm⁻¹): 3377, 3357, 3273, 3178, 2868, 1747, 1724, 1650. ¹H NMR (400 MHz, dmso) δ 7.62-7.51 (m, 2H), 7.42-7.31 (m, 2H), 4.59 (td, J=11.2, 4.9 Hz, 1H), 4.17 (s, 2H), 4.06 (t, J=8.9 Hz, 1H), 3.97-3.92 (m, 2H), 3.85 (dd, J=8.9, 6.4 Hz, 1H), 3.75-3.62 (m, 2H), 2.81 (qd, J=13.7, 5.0 Hz, 2H).

7b)

Prepare a solution of 368 g of (S)-4-[4-(5-{[(4-chloro-benzylidine)-amino]-methyl}-2-oxo-oxazolidine-3-yl)-phenyl]-morpholin-3-one 23a (0.89 mol) in 2760 mL of toluene and 920 mL of isobutylamine. Heat at 80-85° C. and maintain for 6 hours. Cool at room temperature and distil vacuum isobutylamine and part of toluene. At 20° C. filter and wash twice with 500 mL of toluene at room temperature. Dry at 50° C. in a vacuum oven to obtain 254 g of the intended product (yield 98.5%).

7c)

Prepare a solution of 1 g of (S)-4-[4-(5-{[(4-chloro-benzylidine)-amino]-methyl}-2-oxo-oxazolidine-3-yl)-phenyl]-morpholin-3-one 23a in 10 mL of propylamine. Heat at reflux and maintain for 15 hours. The intended product is obtained with a transformation above 98% (assayed by HPLC).

7d)

Prepare a solution of 36 g of (S)-4-[4-(5-{[(4-chloro-benzylidene)-amino]-methyl}-2-oxo-oxazolidine-3-yl)-phenyl]-morpholin-3-one 23a in 180 mL of toluene and 180 mL of isobutylamine. Heat at reflux and maintain for 3 hours. Cool at room temperature and remove the solvent at reduced pressure. Stir the residue obtained at less than 10° C. in 108 mL of toluene. Filter and wash with 50 mL of toluene and then with 20 mL of toluene. After drying at 50° C. in a vacuum oven 25.19 g of the intended product are obtained (yield 99.7%).

### Example 8

### Obtaining (S)-5-chloro-N-((2-oxo-3-(4-(3-oxomorpholin)phenyl)oxazolidine-5-il)methyl)thiophene-2-carboxamide (RVX)

### a) Obtaining 5-chloro-thiophene-2-carbonyl chloride (7)

Mix 2 g of 5-chloro-thiophene-22-carboxylic acid (6) (12 mmol) and 31 mL of thionyl chloride (422 mmol) and heat at reflux for 2 h. Evaporate at reduced pressure the excess thionyl chloride obtaining a brown oil that is used in the next synthesis step without subsequent purification.

### b1) Obtaining Rivaroxaban from 23a (23, R=4-Cl)

Mix 0.25 g of (S)-4-[4-(5-{[(4-chlorobenzylidine)-amino]-methyl}-2-oxo-oxazolidine-3-yl)-phenyl]-morpholin-3-one (23a) (0.6 mmol), 19 mL of water, and 19 mL of AcOEt. Then add 0.1 mL of 37% HCl in water (1.2 mmol) and stir at room temperature for 2 hours. Separate the phases. Extract the aqueous phase with 20 mL of AcOEt. Take the aqueous phase to pH 7-8 adding 1N NaOH. Then add 0.11 g of 5-chloro-thiophene-2-carbonyl chloride (7) (0.6 mmol) dissolved in 19 mL of dichloromethane. Stir at room temperature for 4 hours, maintaining the pH between 7 and 8 by addition of 1 N NaOH. Separate the phases. Extract the aqueous phase with 20 mL of dichloromethane. Join the organic phases, dry with MgSO₄, filter, and remove the solvent at reduced pressure. Suspend the raw matter in hexane (3 mL). Filter the solid obtained and vacuum dry at room temperature, obtaining 0.22 g (83% yield) of a white solid that corresponds to the intended product. Mp=220-225° C. IR (cm⁻¹): 3351; 1754.2, 1645.23, 1630.21. ¹H NMR (500 MHz, CDCl₃) δ 7.56 (d, J=8.9 Hz, 2H), 7.34 (d, J=8.9 Hz, 2H), 7.29 (d, J=4.0 Hz, 1H), 6.90 (d, J=4.0 Hz, 1H), 6.49 (t, J=6.0 Hz, 1H), 4.85 (ddd, J=9.4, 7.8, 4.6 Hz, 1H), 4.34 (s, 2H), 4.10 (t, J=9.0 Hz, 1H), 4.06-4.01 (m, 2H), 3.92-3.87 (m, 1H), 3.84 (dd, J=9.3, 6.8 Hz, 1H), 3.77-3.70 (m, 3H). MS: m/z=458 (M+Na), 436 (M), 893 (2M+Na), 1330 (3M+Na). [α]_{D}=α/c I=−29.35±0.93° g⁻¹ mL dm⁻¹ (c=0.295 g/100 mL in DMSO).

### b2) Obtaining Rivaroxaban from 23b (23, R═H)

Mix 87 mg of (S)-4-(4-{5-[(benzylidene-amino)-methyl]-2-oxo-oxazolidine-3-yl}-phenyl)-morpholin-3-one (23b) (0.23 mmol), 7 mL of water, and 7 mL of AcOEt. Then add 0.04 mL of 37% HCl in water (0.46 mmol) and stir at room temperature for 2 hours. Separate the phases. Extract the aqueous phase with 20 mL of AcOEt. Take the aqueous phase to pH 7-8 adding 1N NaOH. Then add 42 mg of 5-chloro-thiophene-2-carbonyl chloride (7) (0.23 mmol) dissolved in 8 mL of dichloromethane. Stir at room temperature for 2 hours. Separate the phases. Extract the aqueous phase with 20 mL of dichloromethane. Join the organic phases, dry with MgSO₄, filter, and remove the solvent at reduced pressure. Suspend the raw matter in hexane (3 mL). Filter the solid obtained and vacuum dry at room temperature, obtaining 72 mg (72% yield) of a white solid that corresponds to the intended product.

### b3) Obtaining Rivaroxaban from (S)-4-[4-(5-aminomethyl-2-oxo-oxazolidine-3-yl)phenyl]morpholin-3-one (5)

b3.a)

Dissolve 0.145 g of (S)-4-[4-(5-aminomethyl-2-oxo-oxazolidine-3-yl)phenyl]morpholin-3-one (5) (0.5 mmol) in 4 mL of dichloromethane. Add 0.1 mL of triethylamine (0.75 mmol) and then 0.1 g of 5-chlorothiophene-2-carbonyl chloride (7) (0.55 mmol) dissolved in 1 mL of dichloromethane. Stir at room temperature for 3 h. Add 10 mL of water and 10 mL of dichloromethane, separate the organic phase and extract again the aqueous phase with other 10 mL of dichloromethane. Join the organic phases, dry with anhydrous Na₂SO₄, filter and remove the solvent at reduced pressure. 209 mg (96% yield) of whitish solid are obtained, corresponding to the intended product.

b3.b)

Dissolve 0.175 g of (S)-4-[4-(5-aminomethyl-2-oxo-oxazolidine-3-yl)phenyl]morpholin-3-one (5) (0.6 mmol) in 5 mL of THF. Add 0.13 mL of triethylamine (0.9 mmol) and then 0.12 g of 5-chlorothiophene-2-carbonyl chloride (7) (0.66 mmol) dissolved in 1 mL of THF. Stir at room temperature for 3 h. Add 10 mL of water and 10 mL of dichloromethane, separate the organic phase and extract again the aqueous phase with other 10 mL of dichloromethane. Join the organic phases, dry with anhydrous Na₂SO₄, filter and remove the solvent at reduced pressure. 262 mg (100% yield) of a whitish solid are obtained corresponding to the intended product.

b3.c)

Mix 85 g of (S)-4-[4-(5-aminomethyl-2-oxo-oxazolidine-3-yl)phenyl]morpholin-3-one (5) (0.29 mol), 212.5 mL of water, 170 mL of acetone, and 68 mL of toluene. Then add 46.2 g (0.44 mol) of sodium carbonate. Heat the mixture at 55-60° C. At this temperature add in 15 min 72.4 g of 5-chlorothiophene-2-carbonyl chloride (7) (0.40 mol) dissolved in 272 mL of toluene. Stir at 55-60° C. for 5 hours. Cool at 0-5° C., stir at this temperature for 1 hour and filter. Wash twice with 170 mL of toluene and twice with 170 mL with water. 138 g of wet raw matter are obtained, which represent 114.5 g of dry raw matter (yield 90%).

Recrystallise 137 g of raw wet rivaroxaban in 915 mL of acetic acid and take to 90° C. to dissolve the raw matter. Cool at 20° C. and stir for 1 hour at this temperature. Filter and wash three times with 343.5 mL of water. Dry at 50° C. with vacuum to obtain 99.6 g of recrystallized product (yield 87%).

1-20. (canceled)

21. A procedure for obtaining a thiophene-2-carboxamide of formula RVX 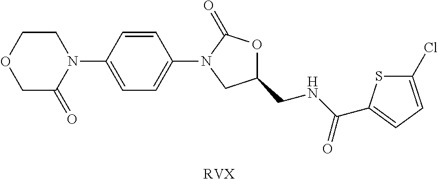
comprising the following steps:
(i) fragmentation of the N═C bond of a compound of formula 23 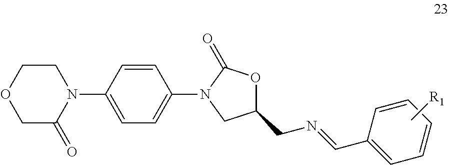
by reaction of compound 23 with a primary amine, in an optional solvent medium;
where R₁ is selected among hydrogen, halogen, and (C₁-C₆)alkyl; and
(ii) acylation of the resulting intermediate of formula 5 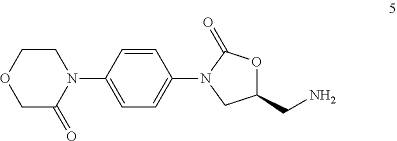 [SMILES: NC[C@H]1CN(c2ccc(N3CCOCC3=O)cc2)C(=O)O1]
with the acid chloride of formula 7 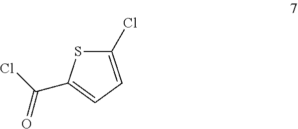
in a solvent medium, in the presence of a base.

22. A procedure according to claim 21, wherein the primary amine is selected from the group consisting of methylamine, ethylamine, propylamine, and isobutylamine.

23. A procedure according to claim 21, where the optional solvent medium is a ester, an ether, an aromatic hydrocarbon, an aliphatic hydrocarbon, a ketone, an alcohol, a halogenated hydrocarbon or water or mixtures thereof.

24. A procedure according to claim 21, where the solvent medium of step (ii) is an ester, an ether, an aromatic hydrocarbon, a ketone, a halogenated hydrocarbon or water, or mixtures thereof, and the base is triethylamine, diisopropylethylamine, sodium hydroxide, potassium hydroxide, sodium carbonate, potassium carbonate, sodium bicarbonate, potassium bicarbonate, calcium hydroxide, calcium carbonate, or calcium bicarbonate or mixtures thereof.

25. A procedure according to claim 24, where the solvent medium of step (ii) is ethyl acetate, methyl acetate, isopropyl acetate, tetrahydrofuran, dioxane, diethyl ether, methyl t-butyl ether, diisopropyl ether, dibutyl ether, toluene, o-xylene, m-xylene, p-xylene, acetone, methyl ethyl ketone, methyl isobutyl ketone, dichloromethane, dichloroethane or water or mixtures thereof.

26. A procedure according to claim 21, where R₁ is hydrogen or 4-chloro.

27. Procedure for obtaining a compound of formula 23 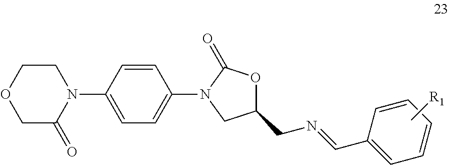
where R₁ is selected among hydrogen, halogen, and (C₁-C₆)alkyl, comprising the reaction of a compound of formula 19 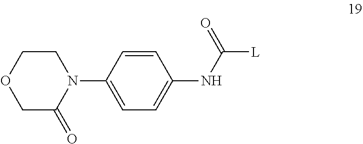
where L is a leaving group, with a compound of formula 22 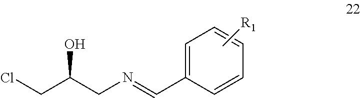
where R₁ has the same meaning as in 23, in the presence of a base and in a solvent medium.

28. A procedure according to claim 27, where the leaving group L is O—R₂, where in turn R₂ is (C₁-C₁₅)alkyl, aryl, aryl-(C₁-C₄)alkyl, heteroaryl, or heteroaryl-(C₁-C₄)alkyl; and
a) aryl is phenyl or naphthyl, that may be optionally substituted;
b) heteroaryl is a monocyclic aromatic ring of 5 or 6 members or bicyclic of 8 to 10 members, containing 1 to 4 heteroatoms selected independently among nitrogen, oxygen and sulphur, and that can be optionally substituted;
the expression “optionally substituted” is defined as the optional presence of 1 to 3 substituents selected independently among (C₁-C₄)alkyl, halogen, (C₁-C₄)alkoxy, —CF₃, —CN, —NO₂, —OH, —COR′, —OCOR′, —CO₂R′, —CONR′R″, —NR′R″, —NR″CO₂R′, —SOR′ and —SO₂R′, where R′ and R″ independently represent hydrogen, (C₁-C₄)alkyl, phenyl, or naphthyl.

29. A procedure according to claim 27, where the base is lithium tert-butoxide or lithium tert-amoxide and the solvent medium comprises an ether, an aliphatic hydrocarbon, a halogenated hydrocarbon or a nitrile, or mixtures thereof.

30. A procedure according to claim 29, where the solvent medium comprises tetrahydrofuran, dioxane, diethyl ether, methyl t-butyl ether, diisopropyl ether, dibutyl ether, heptane, hexane, dichloromethane, dichloroethane, acetonitrile, propionitrile, butyronitrile, or benzonitrile, or mixtures thereof.

31. A procedure for obtaining a thiophene-2-carboxamide of formula RVX 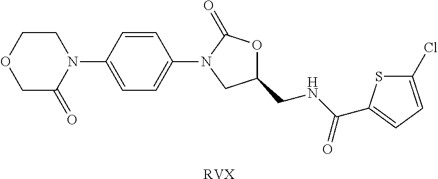
comprising the following steps:
(i) reaction of a compound of formula 19 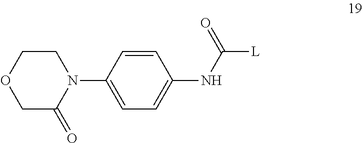
where L is a leaving group, with a compound of formula 22 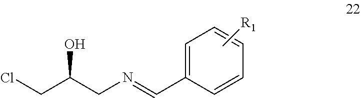
where R₁ is selected among hydrogen, halogen and (C₁-C₆)alkyl, in the presence of a base and in a solvent medium;
(ii) fragmentation of the N═C bond of a compound formed in step (i), of formula 23 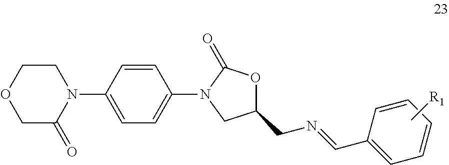
by reaction of compound 23 with a primary amine, in an optional solvent medium;
where R₁ has the same meaning as in 22; and
(iii) acylation of the intermediate resulting of formula 5 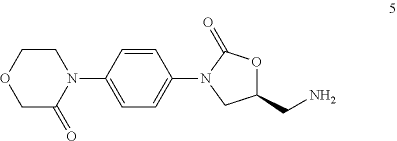 [SMILES: NC[C@H]1CN(c2ccc(N3CCOCC3=O)cc2)C(=O)O1]
with the acid chloride of formula 7 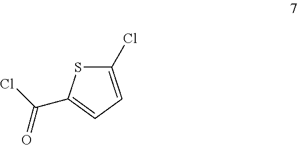
in a solvent medium, in the presence of a base.

32. A procedure according to claim 31, where the leaving group L is O—R₂, where in turn R₂ is (C₁-C₁₅)alkyl, aryl, aryl-(C₁-C₄)alkyl, heteroaryl, or heteroaryl-(C₁-C₄)alkyl; and
a) aryl is phenyl or naphthyl, that may be optionally substituted;
b) heteroaryl is a monocyclic aromatic ring of 5 or 6 members or bicyclic of 8 to 10 members, containing 1 to 4 heteroatoms selected independently among nitrogen, oxygen and sulphur, and that can be optionally substituted;
the expression “optionally substituted” is defined as the optional presence of 1 to 3 substituents selected independently among (C₁-C₄)alkyl, halogen, (C₁-C₄)alkoxy, —CF₃, —CN, —NO₂, —OH, —COR′, —CO₂R′, —CONR′R″, —NR′R″, —NR″CO₂R′, —SOR′ and —SO₂R′, where R′ and R″ independently represent hydrogen, (C₁-C₄)alkyl, phenyl, or naphthyl.

33. A procedure according to claim 31, where R₁ is hydrogen or 4-chloro.

34. A procedure according to claim 31, wherein the primary amine is selected from the group consisting of methylamine, ethylamine, propylamine, and isobutylamine.

35. A procedure according to claim 34, where the optional solvent medium is a ester, an ether, an aromatic hydrocarbon, an aliphatic hydrocarbon, a ketone, an alcohol, a halogenated hydrocarbon or water, or mixtures thereof.

36. A procedure according to claim 31, where
a) in step (i) the base is lithium tert-butoxide or lithium tert-amoxide and the solvent medium is constituted by an ether, an aliphatic hydrocarbon, a halogenated hydrocarbon or a nitrile, or mixtures thereof; and
c) in step (iii) the solvent medium is an ester, an ether, an aromatic hydrocarbon, a ketone, a halogenated hydrocarbon or water, or mixtures thereof and the base is triethylamine, diisopropylethylamine, sodium hydroxide, potassium hydroxide, sodium carbonate, potassium carbonate, sodium bicarbonate, potassium bicarbonate, calcium hydroxide, calcium carbonate, or calcium bicarbonate or mixtures thereof.

37. A procedure according to claim 36, where:
a) in step (i) the solvent medium is constituted by tetrahydrofuran, dioxane, diethyl ether, methyl t-butyl ether, diisopropyl ether, dibutyl ether, heptane, hexane, dichloromethane, dichloroethane, acetonitrile, propionitrile, butyronitrile, or benzonitrile, or mixtures thereof; and
c) in step (iii) the solvent medium is ethyl acetate, methyl acetate, isopropyl acetate, tetrahydrofuran, dioxane, diethyl ether, methyl t-butyl ether, diisopropyl ether, dibutyl ether, toluene, o-xylene, m-xylene, p-xylene, acetone, methyl ethyl ketone, methyl isobutyl ketone, dichloromethane, dichloroethane or water, or mixtures thereof.

38. A compound of formula 23 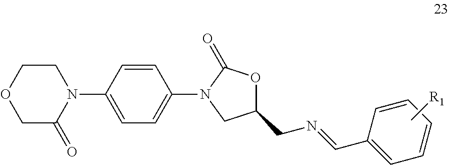
where R₁ is selected among hydrogen, halogen, and (C₁-C₆)alkyl.

39. A compound according to claim 38, where R₁ is hydrogen or 4-chloro.

40. The use of compound according to claim 38 for the synthesis of rivaroxaban (RVX).
